(19) United States
(12) Patent Application Publication
(10) Pub. No.: US 2023/0364377 A1
(43) Pub. Date: Nov. 16, 2023

(54) System And Methods For Vestibular Stimulation For Modulating Sleep

(71) Applicant: Massachusetts Institute of Technology, Cambridge, MA (US)

(72) Inventors: Abhinandan JAIN, Cambridge, MA (US); Pattie MAES, Cambridge, MA (US)

(73) Assignee: Massachusetts Institute of Technology, Cambridge, MA (US)

(21) Appl. No.: 18/316,324

(22) Filed: May 12, 2023

Related U.S. Application Data
(60) Provisional application No. 63/340,971, filed on May 12, 2022.

(51) Int. Cl. A61M 21/02

(52) U.S. Cl. CPC A61M 21/02; A61M 2021/0027

(57) ABSTRACT

Described are systems, devices and methods that use vestibular stimulation to control and improve sleep. The systems, devices and methods described herein are the first to use either one or more than one method of vestibular stimulation from a plurality of different vestibular stimulations (e.g., electrical, thermal, acoustic, optical or tactile) to create illusory motions for affecting sleep. The described systems and methods are the first to utilize an algorithm to determine which stimulation technique to use, when to use a particular stimulation technique, and the unique characteristics to include in the stimulation technique.

### CROSS REFERENCE SECTION

This application claims benefit of U.S. Provisional Application No. 63/340,971 filed on May 12, 2022.

### BACKGROUND

Many adults (by some estimates 50-70 million adults) in the United States have chronic sleep and wakefulness disorders. Sleep disorder issues range from insomnia, bad sleep quality, less deep sleep which in turn affect the waking cognitive performance and long-term health of individuals.

Rocking or rhythmic lateral passive movements are known to promote sleep in infants and adults. Rocking has been shown to modulate sleep onset and duration, improve deep sleep and promote markers of sleep quality such as slow waves and sleep spindles. For example, mechanical rocking of a bed has been shown to improve sleep quality by advancing sleep onset and promoting deep sleep. Consequently, systems generating mechanical motion integrated in mattresses have been proposed. Such mechanical systems, however, are limited due to their cumbersome and mechanical nature.

### SUMMARY

In accordance with one aspect of the concepts disclosed herein, described are systems, devices and methods that use vestibular stimulation to control and improve sleep. The systems, devices and methods described herein are the first to use either one or more than one method of vestibular stimulation selected from a plurality of different vestibular stimulations (e.g., electrical, thermal, acoustic, optical or tactile) to create illusory motions for affecting sleep. That is, the described systems, devices and methods select one or more types of vestibular stimulation from a variety of available choices of vestibular stimulation to create illusory motions for affecting sleep. The systems and methods described herein utilize a process or algorithm to determine one or more of: (a) which stimulation method to use (e.g., one or more of electrical, thermal, acoustic, optical or tactile stimulation to use); (b) when to use a particular stimulation method; and (c) what the unique characteristics (e.g. waveform, intensity, frequency and direction) of the stimulation (e.g. electrical, thermal, acoustic, optical or tactile) should be used.

The system described herein is believed to be the first closed-loop system capable of detecting sleep onset and sleep stages of a user and intervene (e.g., provide non-mechanical stimulation) to advance or delay sleep onset and modulate a duration (a length of time) of one or more sleep stages by either increasing and/or decreasing the duration of the sleep stage.

In accordance with a further aspect of the concepts disclosed herein, a system for improving a sleep pattern of a user learns what stimulation methods and what durations, strengths, patterns, etc., of stimulation best work for one unique user. This may be accomplished, for example, by learning optimal sleep patterns of a user and then monitoring sleep characteristics/changes of the user in response to the user being exposed to particular ones (or particular patterns/sequences) of vestibular stimulations (e.g., electrical, thermal, acoustic, optical or tactile) as well as the characteristics (e.g., waveform, intensity, frequency and direction) of vestibular stimulations applied to the user. Thus, the system is adaptive in that it is capable of changing the type of vestibular stimulations (e.g., electrical, thermal, acoustic, optical or tactile) to provide to a user and/or changing the unique characteristics (e.g., waveform, intensity, frequency and direction) of the stimulation and/or changing a pattern (or sequence) of vestibular stimulations to apply to a user.

In accordance with a still further aspect of the concepts disclosed herein, in embodiments, a vestibular stimulation system comprises an optional sleep stage monitoring system/component configured to monitor sleep characteristics/changes of a user in response to the user being exposed to particular ones (or particular patterns/sequences) of vestibular stimulations (e.g. electrical, thermal, acoustic, optical or tactile) as well as the characteristics (e.g. waveform, intensity, frequency and direction) of vestibular stimulations applied to the user. Such a sleep monitoring system may, for example, detect and provide information which allows the vestibular stimulation system to learn user sleep patterns (and ideally, optimal sleep patterns of a user) and then adjust/switch or otherwise change particular vestibular stimulations (or particular patterns/sequences of vestibular stimulations) to which a user is exposed and/or adjust/switch or otherwise change particular characteristics (e.g. waveform, intensity, frequency and direction) of vestibular stimulations applied to the user.

In accordance with a still further aspect of the concepts disclosed herein, described are methods for detecting and modulating sleep onset and sleep depth by creating illusory motions in yaw, pitch and roll axes. In embodiments, the methods may be implemented via one or more processors executing one or more instructions. In embodiments, the one or more instructions may be in the form of instructions sets, software including application software or any form of computer program. The illusory motions in yaw, pitch and roll axes are created in a user by exposing (or applying) the user to particular vestibular stimulations (e.g., electrical, thermal, acoustic, optical or tactile) having particular characteristics (e.g., waveform, intensity, frequency and direction).

In embodiments, the concepts, systems and methods detect one or more sleep stages and sleep characteristics using one or more wearable or ambient sensors. In response to the detected one or more sleep stages, the disclosed the concepts, systems and methods provide non-mechanical, non-invasive, adaptive and personalized vestibular stimulation by means of one or more electrical, thermal, acoustic, optical or tactile techniques to modulate the sleep onset, control durations of sleep stages, modulate deep sleep and improve overall sleep quality of a user.

One significant difference between the concepts described herein and prior art techniques is that systems and methods described herein use a non-mechanical means of inducing the illusion of motion in a user as the user goes to sleep (i.e., a user's sleep quality is influenced using static vestibular stimulation techniques).

In embodiments, systems and methods described herein may be implemented as a wearable article which allows for ease of use with plug-and-play mechanisms and customization of parameters such as intensity, frequency, direction and dynamics of rocking without using any moving parts. Along with stimulation techniques, the concepts described herein may also include motion and optical sensors configured to capture sleep dynamics such as sleep physiology, onset, duration and depth to make it a closed-loop system. This also enables the system to learn from sleep patterns and thus adapt to different sleep patterns and be personalized for each user.

Different from prior art techniques, the concept of using static vestibular stimulation techniques for influencing sleep quality as described herein may be implemented as a system, device method, and/or computer programs. In embodiments, the concepts, systems, devices and methods described herein are directed toward a non-mechanical and static wearable system for creating illusory sensations of motion, which are automatically determined by the user's sleep patterns to improve and optimize sleep quality.

In contrast to prior art systems and methods, the concepts, systems, devices and methods described herein target user sleep quality and also targets full night sleep and small naps via the use of vestibular stimulation to generate illusory motion and effect sleep. The concepts, systems, devices and methods described may be configured to modulate sleep quality.

### DESCRIPTION OF THE SEVERAL VIEWS OF THE DRAWINGS

The manner and process of making and using the disclosed embodiments may be appreciated by reference to the figures of the accompanying drawings. It should be appreciated that the components and structures illustrated in the figures are not necessarily to scale, emphasis instead being placed upon illustrating the principals of the concepts described herein. Like reference numerals designate corresponding parts throughout the different views. Furthermore, embodiments are illustrated by way of example and not limitation in the figures, in which:

FIG. 1 is a block diagram of an example vestibular simulation sleep system;

FIGS. 2A-2C are diagrammatic views of sensor and stimulation devices coupled to a user's head;

FIG. 3 is a flow diagram illustrating a process for providing non-mechanical, non-invasive, and adaptive vestibular stimulation;

FIG. 4 is a flow diagram of an example process for detecting and modulating sleep onset and sleep depth by creating illusory motions in one or more of yaw, pitch and roll axes and providing non-mechanical, non-invasive, adaptive and personalized vestibular stimulation;

FIG. 5 is a flow diagram of an example process for personalizing stimulation parameters for a user;

FIG. 6 is a flow diagram of an example process for adapting stimulation based in response to changes in sleep stages of a user;

FIG. 7 is a flow diagram of an example process for improving overall sleep patterns for a user;

FIGS. 8A, 8B are plots illustrating a sample stimulation profile for wake to N1; and

FIG. 9 is a diagram illustrating features, sleep stages of a user in addition to and vestibular stimulation provided to the use.

### DETAILED DESCRIPTION

Although reference may sometimes be made herein to particular devices (e.g., sensors), it should be appreciated that other devices having similar functional and/or structural properties may be substituted where appropriate, and that a person having ordinary skill in the art would understand how to select such devices and incorporate them into embodiments consistent with the concepts, systems and methods set forth herein without deviating from the scope of the teachings provided herein.

Further, various example embodiments of the concepts, systems, devices, structures and techniques sought to be protected are described herein with reference to the related drawings and it should be appreciated that alternative embodiments can be devised without departing from the scope of the concepts, systems and methods described herein.

In general overview, the concepts, systems and methods disclosed herein detect one or more sleep stages and sleep characteristics of a user using one or more wearable or ambient sensors. In response to the detected one or more sleep stages, the disclosed the concepts, systems and methods provide non-mechanical, non-invasive, adaptive vestibular stimulation by means of one or more electrical, thermal, acoustic, optical or tactile techniques to modulate the sleep onset, control durations of sleep stages, modulate deep sleep and improve overall sleep quality of a user. In embodiments, the concepts, systems and methods provide personalized) vestibular stimulation

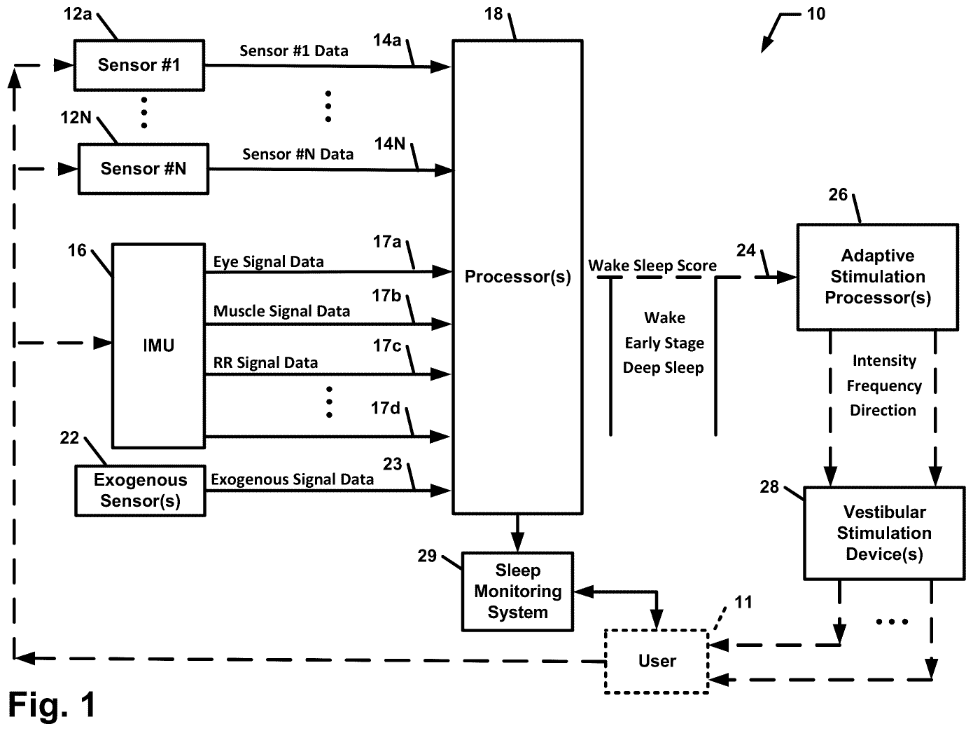

Referring now to FIG. 1, an example of a vestibular stimulation sleep system 10 includes one or more sensors 12a-12N configured to measure parameters of a user 11 (with user 11 here shown in phantom since the user is not properly a part of system 10). The one or more sensors 12a-12N may include, but are not limited to, sensors to measure or detect heart rate (HR) signals, sensors to measure or detect electrical activity of a user's heart (e.g., electrocardiogram (ECG) signals), and sensors to measure or detect electrical activity of a user's brain (e.g., electroencephalogram EEG) signals).

Vestibular stimulation system 10 further includes an inertial measurement unit (IMU) configured to measure various aspects of a user's position (e.g., orientation) and/or movements. NU 16 may comprise one or more sensors (e.g., one or more inertial sensors). NU sensors may include, but are not limited to, one or more of: accelerometer(s) and gyroscope(s). IMU may also comprise sensors to measure characteristics exogenous to a user. Such exogenous characteristics may include environmental characteristics including but not limited to temperature, (e.g., ambient temperature of an environment in which a user a user is disposed), humidity (e.g., humidity of an environment in which a user a user is disposed) and air flow (e.g., air flow of an environment in which a user a user is disposed).

Such exogenous characteristics may also be measured or detected via one or more exogenous sensors 22 (i.e., sensors external to system 10). Such exogenous sensors may include, but are not limited to, room temperature sensors, barometers and humidity sensors.

Data from sensors 12a-12N, 16, 22 may be provided via one or more respective signal paths 14a-14N, 17a-17N, 23 to a processor 18 which processes the signals and associated data in a manner to be described below. It should be appreciated that although processor 18 is illustrated as a single processor in FIG. 1, in practical embodiments, processor 18 may be multiple different processors or processing elements. In embodiments comprising multiple different processors or processing elements some or even all of the different processors or processing elements may be configured to perform a specific function. In embodiments comprising multiple different processors or processing elements some or even all of the different processors or processing elements configured to perform multiple functions.

Further, it is noted that signal paths 14a-14N, 17a-17N, 23 may be wired or wireless signal paths. For example, signals from one or more sensors 12a-12N, 16 may be provided to processor 18 using a short-range wireless technology such as Bluetooth, or any wireless network protocol such as those based upon IEEE 802.11 (Wi-Fi). Other wireless communication paths, connections or technologies, may of course, also be used.

In embodiments, one or more of sensors 12a-12N, 18, 22 may be integrated sensors (e.g., integrated into an apparatus or article worn by user 11 to detect user movements and/or sleep characteristics). In such embodiments, sensors may or may not be in physical contact with a user.

In embodiments, one or more of sensors 12a-12N, 18, 22 may be external sensors (i.e., sensors not in physical contact with a user). Such sensors may include existing ambient sensing systems (e.g., room temperature sensors) configured to provide measured data to system 10 which data may be used (possibly in combination with data from one or more other sensors which may or may not be integrated sensors) to detect user sleep characteristics. For example, in embodiments, the system may comprise a sleep mask, a head wearable, a neck wearable or a sticker configured to contact the user, and into which one, some or all of sensors 12a-12N, 16, 22 may be integrated. In such an embodiment, the integrated sensors may or may not physically contact the user (i.e., may or may not be directly connected to the user.

In embodiments, the system may utilize ambient room or bedside non-contact sensors (e.g., one or more exogenous sensors 22) in which case the vestibular stimulation system may utilize ambient room or bedside non-contact methods

Data from the one or more sensors 12a-12N, 16, 22 (or from a combination of the sensors) may be processed (e.g., fused) in processor 16. It should also be appreciated that some or all of the one or more sensors 12a-12N, the one or more IMU sensors 18 (as well as the IMU processes and algorithms) and the one or more exogenous sensors 22 may be optimized for different sensor configurations, output requirements, and motion constraints. In embodiments, IMU data from multiple inertial sensors may be directly fused by the IMU (rather than by processor 16). Also, in embodiments, IMU 16 may fuse IMU data with data from one or more other sensors 12a-12N, 22 and provide the IMU fused data to processor 16. For example, in embodiments IMU 16 may gather motion data of eyelid, eyebrow and head movements which would translate to eye signals, eye-brow muscle signals and head signals respectively. Such data may be processed to determine sleep characteristics such as sleep onset or sleep stages of a user. IMU 16 or processor 18 may process physiological signals such as heart rate and respiration rate (e.g., as measured using one or more of sensors 12a-12N). Also, IMU 16 may include one or more temperature sensors along with one or more of: one or more acceleration sensors, one or more gyroscopes and one or more magnetic sensors.

Regardless of how processor 16 receives data or what particular type of data is provided thereto, processor 16 processes the data provided thereto (e.g., via the one or more wearable, ambient or IMU sensors) to detect sleep onset and one or more sleep stages and sleep characteristics of a user. In response to the detected sleep onset or one or more sleep stages and/or user sleep characteristics, processor 16 provides a wake sleep score to an adaptive stimulation processor 24. Thus, the combination of sensors and processors provide a means for detecting sleep onset of a user and/or means for detecting a sleep stage of the user.

The wake sleep score is comprised of multiple real time characteristics of user's sleep. For example, the wake sleep score may be determined from one or a combination of: the active sleep stage user is in (N1/N2/N3/REM); duration spent in that sleep stage; sleep related features (e.g., eye movements, sleep spindles, K-Complexes); and physiological signals such as heart or respiration rate and other characteristics, which all are computed from data collected by one or more sensors. In embodiments the wake sleep score may be computed or otherwise determined in accordance with the official sleep scoring manual from the American Academy of Sleep Medicine (AASM) and/or in accordance with known techniques.

Adaptive stimulation processor 26 receives the data provided thereto (e.g., from processor 16 and/or other sources) and provides one or more control signals to one or more stimulation devices 28. Adaptive stimulation processor 26 adapts the stimulation based upon one or more of intensity, frequency or directionality of illusory movements. Such stimulation may be personalized for each individual user.

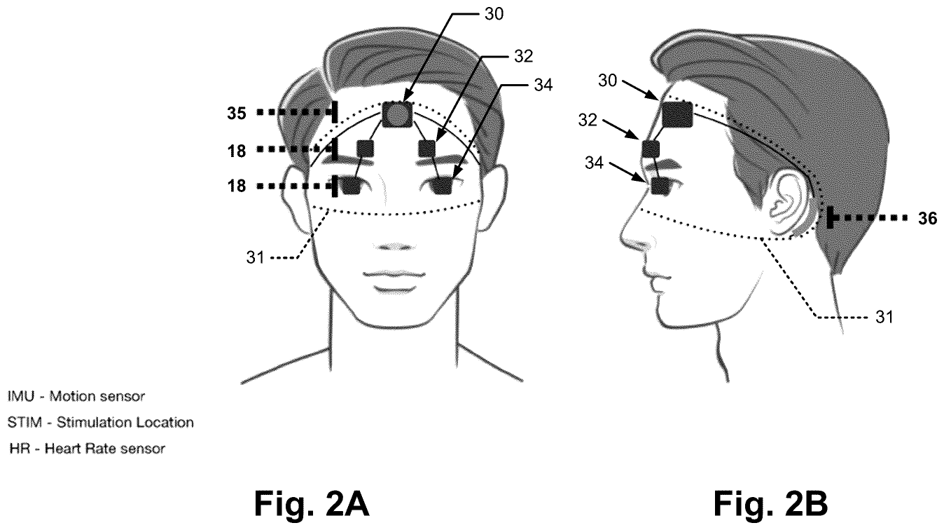

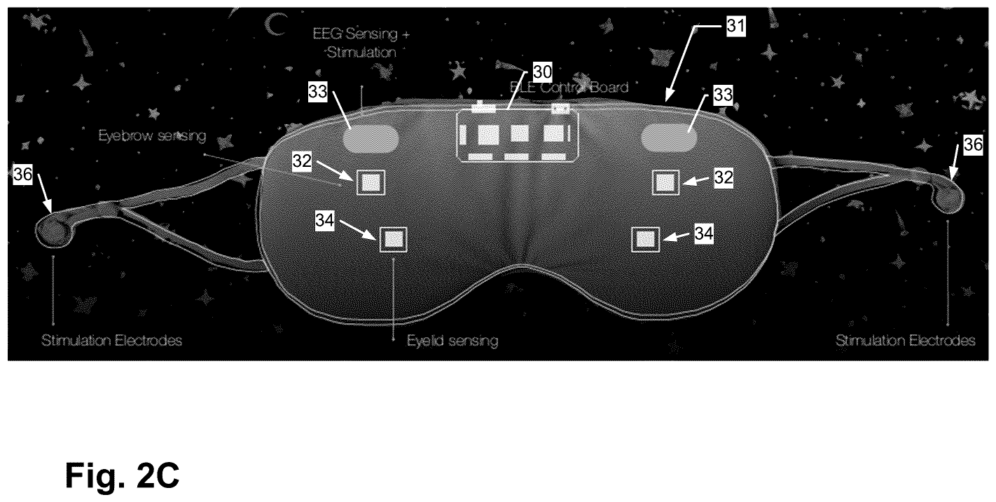

The adaptive stimulation processor receives input features (e.g., from the wake sleep score and/or sensor data) and tunes the output signal characteristics. For example, for helping in sleep onset, the transition between Wake and N1, the stimulator may actively configure the output for a certain frequency (0.5 Hz lateral rocking at maximum Intensity) and then change the output as the user transitions into N1 (reducing to 0.125 Hz at 30% intensity) and then cutoff as user reaches N1. Similarly, signal characteristics may be tuned to assist in transition across other sleep stages. For example, very slow rhythmic rocking (e.g., rocking having a frequency of about 0.05 Hz) may be used in transition from N1 to N2/N3 stage. The decision of assisting in transitions in specific stages and the associated processes could be manually established or be learned from user sleep patterns over time. Learning from user sleep patterns over time may allow the sleep system (e.g., as shown in FIGS. 1 and 2A-2C) to improve (and ideally, optimize) user sleep. Thus, adaptive stimulation processor acts as a means for selecting one or more of a plurality of different types of vestibular stimulation to apply to a user depending upon a detected sleep stage of the user.

The one or more stimulation devices 28 provide non-mechanical, non-invasive, adaptive and personalized vestibular stimulation by means of one or more electrical, thermal, acoustic, optical or tactile signals to modulate the sleep onset, control durations of sleep stages, modulate deep sleep and thereby improve overall sleep quality. In embodiments, the vestibular stimulation device or technique can be through one or a combination of: electrical stimulation (Galvanic Vestibular Stimulation); thermal stimulation (Caloric Vestibular Stimulation); and/or acoustic (Bone Conduction Vestibular Stimulation). Other techniques may, of course, also be used. It is noted and understood that any stimulation techniques which results in a desired effect on a used may be used.

Each stimulation type (e.g., electrical, thermal, acoustic, optical or tactile or other type) may evoke different kinds of responses in a user and can be used depending upon the scenario as well as a desired response time. For example, electrical or galvanic vestibular stimulation provided to a user through the sleep system described herein may activate the otoliths which provide a sense of linear acceleration to a user, and the semicircular canals which provide a sense of angular acceleration to a user. Caloric vestibular stimulation creates convection currents that stimulate the semicircular canals. Bone conduction activates the otolith neurons and provides an alternate pathway to inducing linear acceleration and a sense of gravity. Electrical and bone conduction is suitable for linear accelerations, Caloric for angular acceleration. Electrical works the fastest, while caloric the slowest. The system (e.g., the one or more processors 16 or the one or more adaptive stimulation processors 26) automatically selects the one or more than one stimulation techniques and adjusts its characteristics depending on the user's scenario and needs.

Vestibular stimulation devices 28 modulate sleep onset and sleep depth by providing to a user stimulation signals which create (in the user) illusory motions in one or more of yaw, pitch and roll axes. The type, sequence and characteristic of the stimulation signals delivered to the user via stimulation devices 28 are determined via one or more processors (e.g., processors 18 and/or 26). Thus, one or more vestibular stimulation devices provide a means for applying to the user one or more selected types of vestibular stimulation.

The system may further include an optional sleep stage monitoring system 29 (also sometimes referred to herein as a “sleep monitoring system” or a “sleep monitoring component”). In embodiments, the optional sleep monitoring system may provide input to the processor and data provided to the processer from the sleep monitoring component may be used to provide improved (e.g., more accurate) wake sleep scores.

The optional sleep stage monitoring system is configured to monitor sleep characteristics/changes of a user in response to the user being exposed to particular ones (or particular patterns/sequences) of vestibular stimulations (e.g., electrical, thermal, acoustic, optical or tactile) as well as the characteristics (e.g., waveform, intensity, frequency and direction) of vestibular stimulations applied to the user. Such a sleep monitoring system may, for example, detect and provide information which allows the vestibular stimulation system 10 to learn user sleep patterns (and ideally, optimal sleep patterns of a user) and then adjust/switch or otherwise change particular vestibular stimulations (or particular patterns/sequences of vestibular stimulations) to which a user is exposed and/or adjust/switch or otherwise change particular characteristics (e.g. waveform, intensity, frequency and direction) of vestibular stimulations applied to the user.

By receiving feedback of user sleep patterns and/or characteristics (e.g., from a sleep monitoring system or any other means) processor 16 may process such feedback and learns what stimulation methods and what durations, strengths, patterns, etc., of stimulation best work for one unique user. For example, processor 16 can process the data provided thereto to learn optimal sleep patterns of a user and then monitor sleep characteristics/changes of the user in response to the user being exposed to particular ones (or particular patterns/sequences) of vestibular stimulations (e.g., electrical, thermal, acoustic, optical or tactile) as well as the characteristics (e.g., waveform, intensity, frequency and direction) of vestibular stimulations applied to the user. Processor 16 (or processor 26) may then adapt (or change) the particular type of vestibular stimulations (e.g., electrical, thermal, acoustic, optical or tactile) provided to a user and/or change the unique characteristics (e.g., waveform, intensity, frequency and direction) of the stimulation and/or changing a pattern (or sequence) of vestibular stimulations being applied to a user. Such changes/adaptations may be selected to improve use sleep quality.

Thus, in embodiments, with such feedback the system 10 is a closed-loop system capable of detecting sleep onset and sleep stages of a user and intervening (e.g., provide vestibular stimulation) to advance or delay sleep onset and modulate a duration (a length of time) of one or more sleep stages by either increasing and/or decreasing the duration of the sleep stage. Thus, feedback may include the provision of vestibular stimulation.

Thus, system 10 provides non-mechanical, non-invasive, adaptive and personalized vestibular stimulation via means for providing one or more of: electrical, thermal, acoustic, optical or tactile signals to a user. The one or more of electrical, thermal, acoustic, optical or tactile signals modulate sleep onset, control durations of sleep stages, modulate deep sleep and improve overall sleep quality. The system may use integrated or ambient existing sensing systems to detect sleep characteristics. In this way, the system uses vestibular stimulation to control and improve sleep.

Referring now to FIGS. 2A-2C in which like elements are provided having like reference designations, a control device 30 is coupled to one or more sensors 32, 33 34 and one or more stimulation devices 35, 36 are coupled to a user. The sensors and IMU may be the same as or similar to the sensors 12-12N, 22 and IMU 18 described above in conjunction with FIG. 1.

One or more sensors may detect user heart rate. Some devices may perform multiple functions. Device 35 may, for example, function as both a stimulation device and a heart rate sensor. The sensors may be motion and/or optical sensors configured to capture sleep dynamics such as sleep physiology, onset, duration and depth to make it a closed-loop system. Such sensors may further enable a sleep system (such as sleep system 10 in FIG. 1) to learn from sleep patterns and thus adapt to different sleep patterns and be personalized for each user.

In the example of FIG. 2C, sensor 32 may be a sensor to detect eyebrow movement; sensor 33 may be an EEG sensor; and sensor 34 may detect eyelid movement. Electrodes 36 may be stimulation electrodes.

In general, in response to controls signals provided thereto (e.g., from an adaptive stimulation processor, such as that described above in conjunction with FIG. 1), stimulation devices may emit or otherwise provide one or more of: electrical, thermal, acoustic, optical or tactile stimulation signals. Such signals (either individually or in combination) may help modulate sleep onset, control durations of sleep stages, modulate deep sleep and improve overall sleep quality of a user.

Lateral low frequency rhythmic signals could promote sleep onset and deeper sleep, whereas signal in pitch direction could transition to lighter sleep. Such signals could be personalized for the user depending on their sensitivity to stimulation and their sleep profiles. For example, for a user, transition between Wake and N1, the stimulation could be of output for a certain frequency (0.5 Hz lateral rocking at maximum calibrated Intensity) and the output changes as the user transitions into N1 (reducing to 0.125 Hz lateral rocking at 30% intensity) and then cuts off as user reaches N1.

For transition between N2 and N3, the stimulation could be of very low frequency (0.05 Hz lateral rocking at 20% intensity) to promote deep sleep.

For transition N3 to N1, the stimulation could be pulse at 40% intensity in pitch direction.

For transition wake, the stimulation could be pulse at 100% intensity in pitch direction to creates a motion illusion of pitching forward.

Through combinations of different signal characteristics such as waveform, frequencies, intensities, variety of effects in modulating sleep could be observed.

As illustrated in FIG. 2A-2C some or all of the sensors and/or stimulation devices (sometimes referred to herein as “stim devices”) may be coupled to or integrated in (i.e., be embedded in or physically coupled to) a wearable article 31. In the example embodiment of FIGS. 2A-2C wearable article 31 is illustrated as a sleep mask. Is should, however, be appreciated that in other embodiments, it may be desirable or even necessary to provide wearable article 31 as a hat or other head wearable article, a neck wearable article, a sticker (e.g., any type of adhesive label configured to be attached to a user) or as a combination of apparel and/or stickers. Regardless of the particular manner in which wearable article 31 is implemented, wearable article 31 provides an integrated sensing and stimulation system to a user. The sensors and wearable article 31 may be configured such that one or more of sensors and stimulation devices contacts (either directly or indirectly) and/or are disposed in proximity to the user's skin or other surface of the user. In embodiments, the sleep system may use one or more stimulation devices. In one example embodiment, the sleep system includes four (4) stimulation contact locations (one (1) on each mastoid process and two (2) on forehead with a stim device at each contact point). It is appreciated there is a concomitant relationship between the number of contact points and the amount of fidelity and fine-tuning capability of the sleep system. Thus, a system utilizing more than four (4) contact points (with a stim device at each contact point) may have increased fidelity and fine-tuning capability compared with systems having four (4) or fewer contact points.

A wearable article may allow for ease of use with plug-and-play mechanisms (e.g., hardware modules, software modules or modules comprising a combination of hardware and software) and customization of parameters such as intensity, frequency, direction and dynamics of rocking without using any moving parts. Plug and play mechanisms as additional modules which could be attached or otherwise coupled (either with a wired connection or via a wireless connection) to a base system to increase the functionality and/or improve performance of a base system. For example, in embodiments, an acoustic sensor module to sense breathing or snoring may be coupled to a system. Or additional stimulation devices may be coupled at additional contact locations to improve stimulation fidelity.

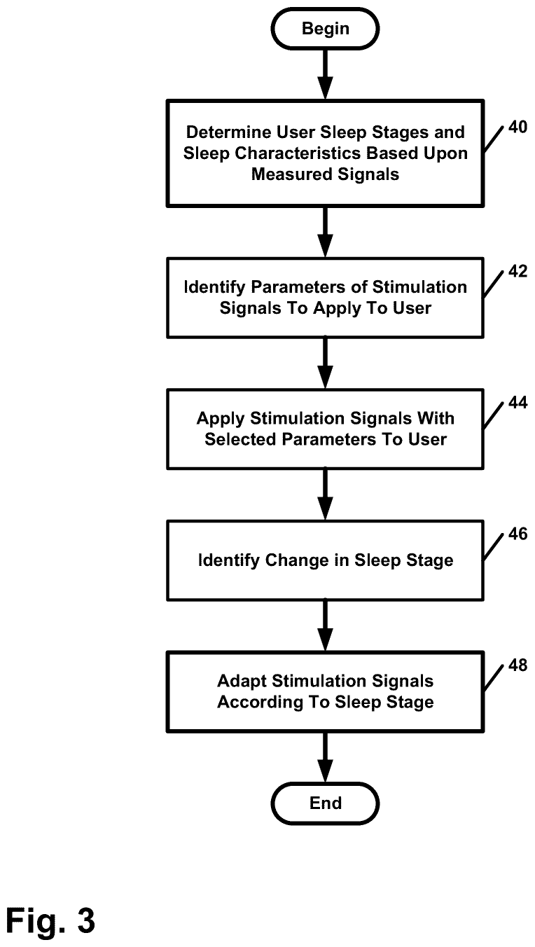

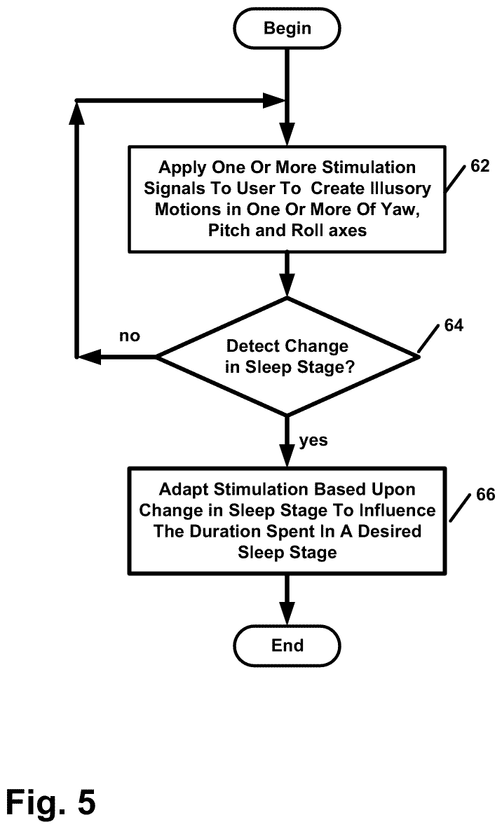

FIGS. 3-5 are flow diagrams showing illustrative processing that can be implemented within system 10 (FIG. 1). Rectangular elements (typified by element 40 in FIG. 3, are herein denoted “processing blocks,” and represent processing instructions (e.g., computer software instructions) or groups of instructions. Diamond shaped elements (typified by element 64 in FIG. 5), are herein denoted “decision blocks,” and represent processing instructions (e.g., computer software instructions), or groups of instructions, which affect the execution of the instructions represented by the processing blocks.

Alternatively, the processing and decision blocks may represent functions performed by functionally equivalent circuits such as a digital signal processor circuit or an application specific integrated circuit (ASIC). The flow diagrams do not depict the syntax of any particular programming language. Rather, the flow diagrams illustrate the functional information one of ordinary skill in the art requires to fabricate circuits or to generate computer software or other processing instructions needed to perform the processing required of a particular apparatus. It should be noted that many routine program elements, such as initialization of loops and variables and the use of temporary variables are not shown. It will be appreciated by those of ordinary skill in the art that unless otherwise indicated, the particular sequence of blocks described is illustrative only and can be varied without departing from the spirit of the concepts, structures, and methods described herein. Thus, unless otherwise stated, the blocks described below are unordered meaning that, when possible, the functions, processes or methods represented by the blocks can be performed in any convenient or desirable order.

Turning now to FIG. 3, a method for improving user sleep begins by determining user sleep stages and characteristics as shown in processing block 40. Such sleep stages and sleep characteristics may be detected using sensors. Such sensors may be wearable or ambient sensors (i.e., the method may use integrated or ambient existing sensing systems to detect sleep characteristics of a user). User sleep stages and characteristics may be determined from measurements of user characteristics including, but not limited to: heart rate; facial movements (including, but not limited to eye and/or eyelid movements, muscle movements); muscle movements; and RR data. Such measurements may be made with one or more sensors such as the sensors described above in conjunction with FIGS. 1-2B.

Based upon the user sleep stages and characteristics, the system identifies parameters of a stimulation signal which may be applied to the user as shown in processing block 42.

After or concurrently with identification of stimulation signal parameters, one or more stimulation signals with selected ones of the parameters (either one, some or all of the parameters) may be applied to the user as shown in processing block 44. The stimulation signals may be applied using one or more of the stimulation devices described above in conjunction with FIGS. 1-2B.

In processing block 44, changes in sleep stages of the user (if any) are identified. If changes in sleep stages of the user are identified, then the stimulation signals applied to the user are appropriately adapted to correspond to the change in sleep stage. In this way, the method uses vestibular stimulation to control and improve sleep. That is, the method provides non-mechanical, non-invasive, adaptive and personalized vestibular stimulation via stimulation signals corresponding to one or more of electrical, thermal, acoustic, optical or tactile signals provided to a user via one or more stimulation devices. The one or more of electrical, thermal, acoustic, optical or tactile signals may be used to modulate one or more of: sleep onset; control durations of sleep stages; modulate deep sleep; and improve overall sleep quality.

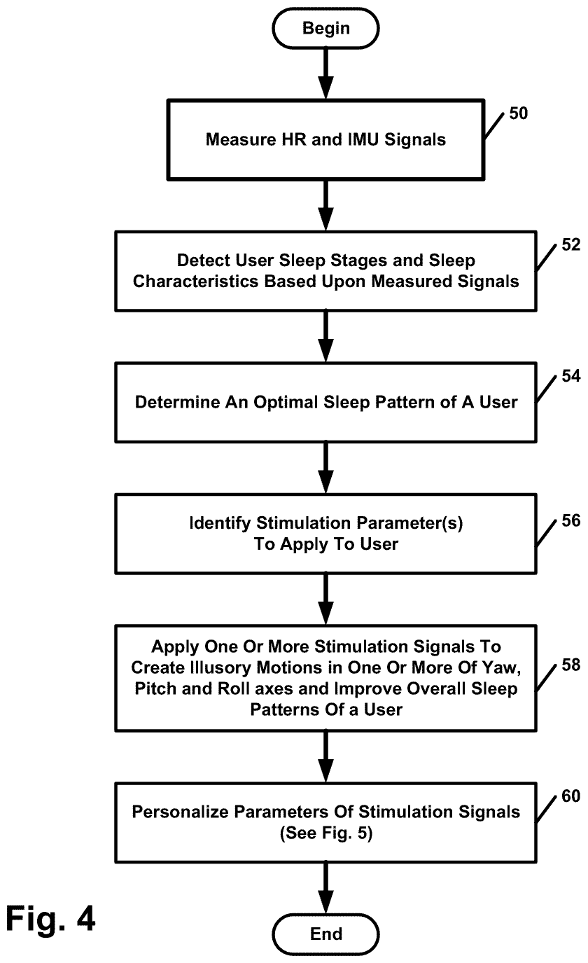

Referring now to FIG. 4, a method for detecting and sleep onset sleep depth and modulating improving user sleep begins by creating illusory motions in one or more of yaw, pitch or roll axes begins in processing block 50 in which HR and IMU signals are measured. Such signals may be measured via wearable or ambient sensors. The HR and IMU signals may include but are not limited to: heart rate; facial movements (including, but not limited to eye and/or eyelid movements, muscle movements); muscle movements; and RR data. Such measurements may be made with one or more sensors such as the sensors described above in conjunction with FIGS. 1-2B.

In processing block 52, user sleep stages are detected or otherwise determined based upon one or more of the measured signals (e.g., one or more of the measured HR and/or IMU signals).

In processing block 54, an optimal sleep pattern of the used is determined. This may be accomplished, for example, by collecting user sleep patterns over multiple nights and correlating them with user reported post sleep quality. This would enable the system to find optimum light or deep sleep durations that give the user the best quality of sleep. Further, user optimum sleep could be modeled by combining daily behavioral metrics such as physical activity, user's schedule, user's circadian rhythms etc. and correlating with sleep patterns.

Based upon the determined optimal sleep pattern of the user, one or more stimulation patterns are identified and then applied to the user to improve overall sleep patterns of the user as shown in processing blocks 56, 58. The application of one or more stimulation signals to a user creates, in the user, illusory motions in yaw, pitch and roll axes. This results in the one or more stimulation signals (e.g., one or more of electrical, thermal, acoustic, optical or tactile signals) modulating one or more of: sleep onset; control durations of sleep stages; modulate deep sleep; and thus improves overall sleep quality of the user.

In processing block 60, the parameters of the stimulation signals are personalized for the user. For example, characteristics such as signal frequencies, signal intensities, signal patterns are specifically selected based upon measurements and/or known characteristics of a user.

Referring now to FIG. 5, a method for personalizing one or more stimulation signals (e.g. one or more characteristics of one or more stimulation signals) for a use begins with processing block 62 and decision block 64 which form a loop in which after application of one or more stimulation signals, if a change in a user sleep stage is detected, then processing proceeds to processing block 66 in which one or more stimulation signals are adapted based upon the detected change in a user's sleep stage. The one or more stimulation signals are adapted to influence a duration (i.e., an amount of time) a user spends in a desired sleep stage.

In processing block 60, parameters of stimulation signals are personalized for a user. This may be accomplished, for example, via an iterative approach to determine desired characteristics of the signals to provide desired results (e.g., achieving an optimum sleep pattern for a user). Then other stimulation characteristics could be judged based upon the results of a first few trials.

In this way, the method provides non-mechanical, non-invasive, adaptive and personalized vestibular stimulation by means of electrical, thermal, acoustic, optical or tactile method to modulate the sleep onset, control durations of sleep stages, modulate deep sleep and improve overall sleep quality.

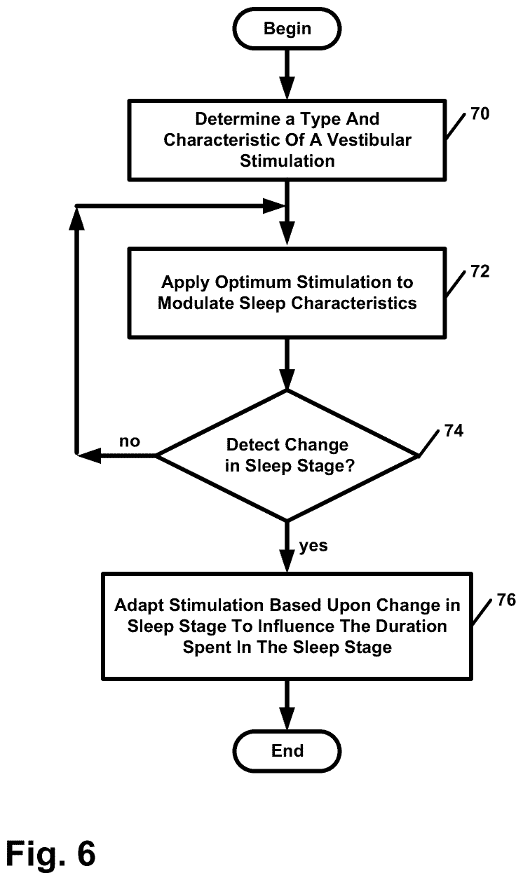

Referring now to FIG. 6, an example process for adapting vestibular stimulation based on changes in sleep stages of a user begins with processing block 70 in which a determination is made of a type and characteristic of a vestibular stimulation to apply to a user. Such determination may be made, for example using input from a sleep monitoring system.

Processing block 72 and decision block 74 form a loop in which after application of optimum stimulation signals to modulate sleep characteristics of a user, if a change in a user sleep stage is detected, then processing proceeds to processing block 76 in which one or more stimulation signals are adapted based upon the detected change in a user's sleep stage. The one or more stimulation signals are adapted to influence a duration (i.e., an amount of time) a user spends in the changed sleep stage.

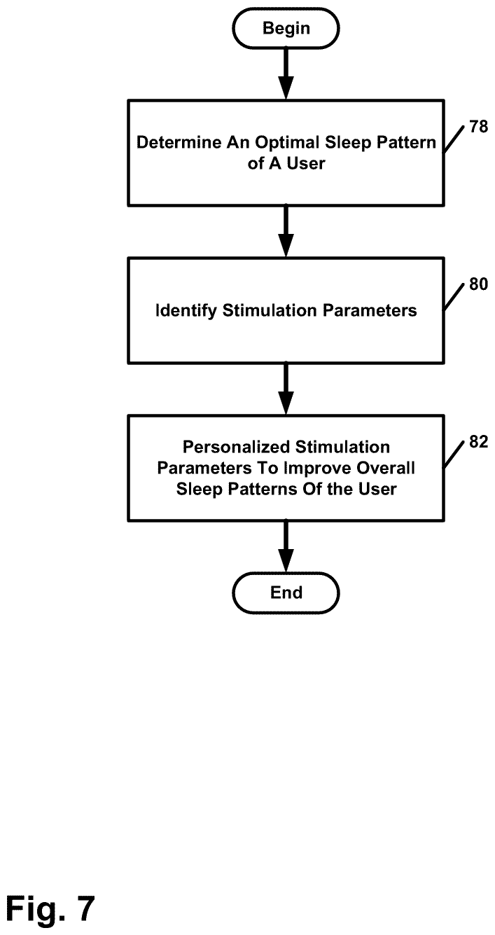

FIG. 7 is a flow diagram of an example process for improving overall sleep patterns for a user. The process begins with processing block 78 in which an optimal sleep pattern of a user is determined. Such determination may be made, for example using input from a sleep monitoring system.

Processing then proceeds to processing block 80 in which parameters (e.g., characteristics) of vestibular stimulation signals to be applied to a user are identified. Stimulation having the identified parameters are applied to a user. Through a monitoring system, stimulation signals and parameters which promote desired sleep characteristics of the user (i.e., the specific/unique user) are identified. A set of stimulation signals and parameters are then identified for the unique user which provide/promote desired sleep characteristics of the user. Such personalized stimulation signals and parameters may be saved (e.g., stored in a memory device) and then applied to the specific user at a later point in time.

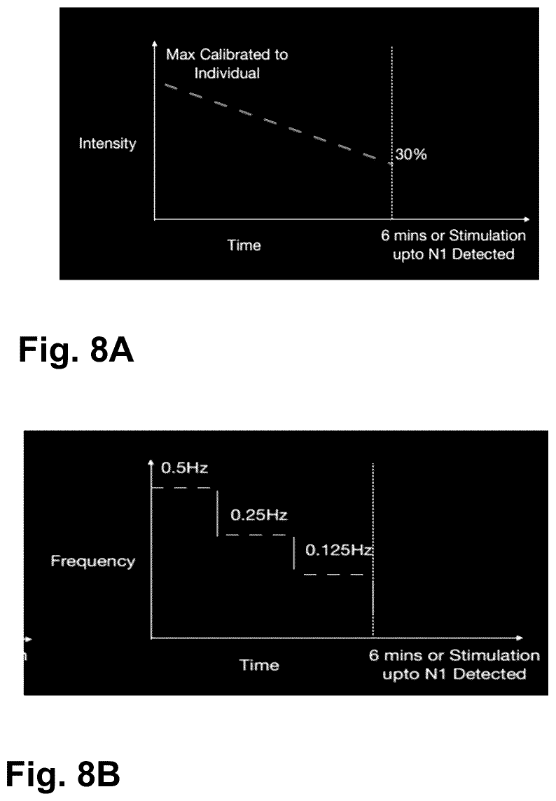

FIGS. 8A, 8B are plots illustrating a sample user stimulation profile for wake to N1. The curve in FIG. 8A illustrates a change in stimulation intensity over time and FIG. 8B illustrates a change in frequency of stimulation over time. As illustrated in the example of FIGS. 8A, 8B, the intensity and frequency of stimulation provided by the sleep system (e.g., the system illustrated in FIG. 1 or FIGS. 2A-2C) decrease as the used transitions from Wake to N1.

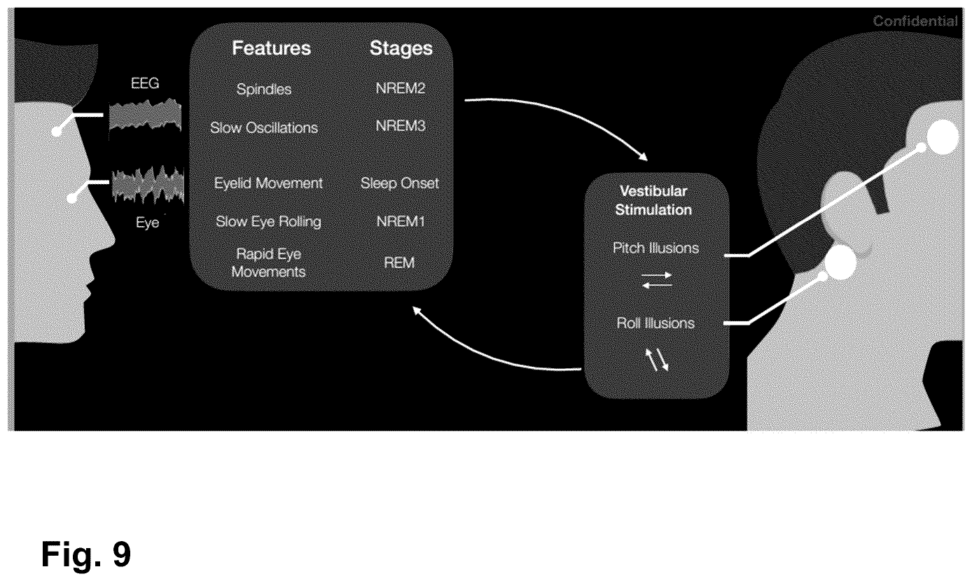

FIG. 9 is a diagram illustrating features and sleep stages of a user as well as vestibular stimulation provided to the user.

As discussed above, systems or devices implementing the concepts and methods described herein may have integrated sensor(s) or use external sensors (e.g., ambient room or bedside non-contact sensors) or systems to detect sleep characteristics.

It is noted that various connections and positional relationships (e.g., over, below, adjacent, etc.) are set forth between elements in the above description and in the drawings. These connections and/or positional relationships, unless specified otherwise, can be direct or indirect, and the described concepts, systems, devices, structures and techniques are not intended to be limiting in this respect. Accordingly, a coupling of entities can refer to either a direct or an indirect coupling, and a positional relationship between entities can be a direct or indirect positional relationship.

As an example of an indirect positional relationship, references in the present description to disposing element (e.g., device) “A” over element “B” include situations in which one or more intermediate elements (e.g., element “C”) is between element “A” and element “B” as long as the relevant characteristics and functionalities of element “A” and element “B” are not substantially changed by the intermediate element(s).

The following definitions and abbreviations are to be used for the interpretation of the claims and the specification. As used herein, the terms “comprises,” “comprising, “includes,” “including,” “has,” “having,” “contains” or “containing,” or any other variation thereof, are intended to cover a non-exclusive inclusion. For example, a system, method, article, or apparatus that comprises a list of elements is not necessarily limited to only those elements but can include other elements not expressly listed or inherent to such system, method, article, or apparatus.

Additionally, the term “exemplary” is used herein to mean “serving as an example, instance, or illustration. Any embodiment or design described herein as “exemplary” is not necessarily to be construed as preferred or advantageous over other embodiments or designs. The terms “one or more” are understood to include any integer number greater than or equal to one, i.e., one, two, three, four, etc. The terms “a plurality” are understood to include any integer number greater than or equal to two, i.e., two, three, four, five, etc.

The term “connection” can include an indirect “connection” and a direct “connection.” As noted above, elements may be connected or coupled through wired or wireless signal paths. For example, signals may be transmitted from one component to another (e.g., between sensors or from a sensor to a controller or processor) using a short-range wireless technology such as Bluetooth, or any wireless network protocol such as those based upon IEEE 802.11 (WiFi). Other wireless communication paths, connections or technologies, may of course, also be used. Wire paths using any type of connector may be used (e.g., any type of sensor connector, any type of USB connector (such as USB-A, USB-B, USB-C, mini-USB, micro-USB); any type of audio connector (e.g., 3.5 MM audio); Lightning connectors; any type of phone connector; any type of HDMI connectors; any type of optical connectors)

References in the specification to “one embodiment, “an embodiment,” “an example embodiment,” etc., indicate that the embodiment described can include a particular feature, structure, or characteristic, but every embodiment can include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

For purposes of the description hereinafter, the terms “upper,” “lower,” “right,” “left,” “vertical,” “horizontal,” “top,” “bottom,” and derivatives thereof shall relate to the described structures and methods, as oriented in the drawing figures. The terms “overlying,” “atop,” “on top, “positioned on” or “positioned atop” mean that a first element, such as a first structure, is present on a second element, such as a second structure, where intervening elements such as an interface structure can be present between the first element and the second element. The term “direct contact” means that a first element, such as a first structure, and a second element, such as a second structure, are connected without any intermediary elements.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

The terms “approximately” and “about” may be used to mean within ±20% of a target value in some embodiments, within ±10% of a target value in some embodiments, within ±5% of a target value in some embodiments, and yet within ±2% of a target value in some embodiments. The terms “approximately” and “about” may include the target value. The term “substantially equal” may be used to refer to values that are within ±20% of one another in some embodiments, within ±10% of one another in some embodiments, within ±5% of one another in some embodiments, and yet within ±2% of one another in some embodiments.

The term “substantially” may be used to refer to values that are within ±20% of a comparative measure in some embodiments, within ±10% in some embodiments, within ±5% in some embodiments, and yet within ±2% in some embodiments. For example, a first direction that is “substantially” perpendicular to a second direction may refer to a first direction that is within ±20% of making a 90° angle with the second direction in some embodiments, within ±10% of making a 90° angle with the second direction in some embodiments, within ±5% of making a 90° angle with the second direction in some embodiments, and yet within ±2% of making a 90° angle with the second direction in some embodiments.

It is to be understood that the disclosed subject matter is not limited in its application to the details of construction and to the arrangements of the components set forth in the following description or illustrated in the drawings. The disclosed subject matter is capable of other embodiments and of being practiced and carried out in various ways. Also, it is to be understood that the phraseology and terminology employed herein are for the purpose of description and should not be regarded as limiting. As such, those skilled in the art will appreciate that the conception, upon which this disclosure is based, may readily be utilized as a basis for the designing of other structures, methods, and systems for carrying out the several purposes of the disclosed subject matter. Therefore, the claims should be regarded as including such equivalent constructions insofar as they do not depart from the spirit and scope of the disclosed subject matter.

Although the disclosed subject matter has been described and illustrated in the foregoing exemplary embodiments, it is understood that the present disclosure has been made only by way of example, and that numerous changes in the details of implementation of the disclosed subject matter may be made without departing from the spirit and scope of the disclosed subject matter.

What is claimed is:

1. A vestibular stimulation sleep system comprising:
sleep onset detection means for detecting sleep onset of a user;
sleep stage detection means for detecting a sleep stage of the user; and
vestibular stimulation selection means for selecting one or more of a plurality of different types of vestibular stimulation to apply to a user depending upon a sleep stage of the user detected by the sleep stage detection means; and
vestibular stimulation means for applying to the user one or more types of vestibular stimulation selected by the selecting means.

2. The vestibular stimulation sleep system of claim 1 wherein the plurality of different types of vestibular stimulation correspond to: electrical stimulation; thermal stimulation; acoustic stimulation; optical stimulation; or tactile stimulation.

3. The vestibular stimulation sleep system of claim 1 further comprising vestibular stimulation changing means for changing a type of vestibular stimulation applied to the user in response to a change in a sleep stage of the user detected by the sleep stage detection means.

4. The vestibular stimulation sleep system of claim 1 further comprising:
sleep stage change detection means for detecting a change in a sleep stage of the user; and
vestibular stimulation change means for changing a type of vestibular stimulation to the user in response to a change in a sleep stage of the user detected by the sleep stage change detection means.

5. The vestibular stimulation sleep system of claim 1 further comprising stimulation characteristic selection means for selecting one or more characteristics of one or more types of vestibular stimulations applied to a user, wherein the one or more characteristics are selected in response to a sleep stage of the user detected by the sleep stage detection means.

6. The vestibular stimulation sleep system of claim 1 further comprising:
sleep stage change detection means for detecting a change in a sleep stage of the user; and
means for selecting one or more characteristics of one or more vestibular stimulations in response to a change in a sleep stage of the user detected by the sleep stage change detection means.

7. The vestibular stimulation sleep system of claim 1 wherein the means for detecting sleep onset of a user comprises one or more of a wearable sensor and an ambient sensor.

8. The vestibular stimulation sleep system of claim 1 the means for detecting a sleep stage of the user comprises one or more of a wearable sensor and an ambient senor.

9. A vestibular stimulation sleep system comprising:
a wearable article;
at least one sensor coupled to the wearable article; and
at least one stimulation device coupled to the wearable article and in communication with at least one of:
a processor configure to receive signals from the at least one sensor and for processing the signals to detect a sleep stage of a user;
a processor for selecting one or more of a plurality of different types of vestibular stimulation to apply to a user in response to the detected sleep stage of the user; and
wherein, in response to a signal provided thereto, the at least one stimulation device applies one or more types of vestibular stimulation to the user.

10. The vestibular stimulation sleep system of claim 9 wherein at least one of the sensors is configured to detect a physiological characteristic of the user.

11. A method comprising:
monitoring one or more sleep characteristics of a user, the sleep characteristics including sleep onset, sleep stages, sleep depth and user's physiology by using wearable or ambient sensors;
determining a user's sleep/wake stage, sleep onset time and duration in a sleep stage; and
applying vestibular stimulation to modulate one or more of the one or more sleep characteristics of the user.

12. The method of claim 11 wherein applying vestibular stimulation to modulate one or more of the one or more sleep characteristics of the user comprises applying stimulation to modulate one or more of:
sleep onset;
one or more sleep stages; or
sleep depth.

13. The method of claim 12 wherein applying stimulation to modulate one or more of:
sleep onset; one or more sleep stages; or sleep depth comprises one or more of:
advancing or delaying sleep onset;
advancing or delaying sleep stages; and
advancing or delaying sleep depth.

14. The method of claim 11 further comprising adapting stimulation based upon a change in sleep stage to influence the duration spent in that sleep stage.

15. The method of claim 11 further comprising:
determining a change in sleep stage from a first sleep stage to a second, different sleep stage; and
in response to a determined change in sleep stage, changing stimulation.

16. The method of claim 15 further wherein changing stimulation comprises changing stimulation to influence the duration spent in the second sleep stage.

17. The method of claim 16 wherein changing stimulation to influence the duration spent in the second sleep stage comprises increasing or decreasing the duration spent in the second sleep stage.

18. The method of claim 11 further comprising learning an optimal sleep pattern of the user based upon the monitored one or more sleep characteristics and the determined sleep/wake stage, sleep onset time and duration in a sleep stage of the user.

19. The method of claim 18 further comprising changing stimulation applied to a user to increase or decrease a duration spent in a sleep stage based upon a learned optimal sleep pattern of the user.

20. The method of claim 18 further comprises using the learned optimal sleep pattern of the user to apply stimulation to modulate advancing or delaying sleep onset; advancing or delaying sleep stages; advancing or delaying sleep depth.
